(19) United States
(12) Patent Application Publication
(10) Pub. No.: US 2012/0123307 A1
(43) Pub. Date: May 17, 2012

(54) Arm brace

(76) Inventor: Ronald Michael Figurski, Kelowna (CA)

(21) Appl. No.: 12/926,422

(22) Filed: Nov. 17, 2010

(51) Int. Cl. A61F 5/00

(52) U.S. Cl. 602/4

(57) ABSTRACT

Disclosed is an apparatus and method for immobilizing the shoulder of a user. The apparatus comprises an elbow cap, a shoulder cap, a first tension member extending between the elbow cap and the shoulder cap and a second tension member extending from the wrist. The method comprises connecting the first tension member between the elbow cap located on an elbow corresponding to the shoulder to be immobilized to the shoulder cap located on an opposed shoulder behind the back of the user and support and maintain the wrist proximate to the user's torso with the second tension member. The method further comprises tightening the first tension member so as to draw the elbow behind a frontal plane of the user's torso proximate to the user's torso.

### BACKGROUND OF THE INVENTION

1. Field of Invention The present invention relates to braces in general and in particular to a method and apparatus for positioning the arm of a wearer.

2. Description of Related Art

Shoulder injuries commonly require immobilization of the shoulder after initial medical treatment has been applied. In particular, for shoulder dislocations, it is common treatment to first return the shoulder to its normal position. Thereafter, the shoulder is immobilized so as to prevent further rotation of the shoulder and to promote healing. Conventional methods of immobilizing the shoulder have been to place the arm corresponding to the injured shoulder in a sling. The use of a sling has also conventionally been utilized post-operatively in shoulder surgeries.

Arm slings 4 typically comprise a sheet of material 6 sized to receive the forearm of the user therein and at least one strap 8 to suspend the sling from the neck of the user as illustrated in FIG. 1. Slings typically locate the forearm of the user in a generally horizontal position across the body of the user. Such slings therefore place the arm in internal rotation (across the body).

Although an arm sling is an accepted and common treatment for dislocated shoulders, recent studies have discovered locating the arm in internal rotation does not greatly assist healing of the injured shoulder and may for some injuries impair healing. Rather, internal shoulder rotation has been shown to pronounce the detachment of the glenoid labrum from the glenoid fossa and thereby reduce the likely hood of the glenoid labrum from healing to the glenoid fossa.

It has also been discovered that placing the arm in external rotation (with the forearm angled away from the body) has been more effective reopposing the glenoid labrum to the glenoid rim which is required for proper healing of the injured shoulder. Previous proposals for locating the injured arm in external rotation require rotating the forearm to be angled away from the body of the user. It will be appreciated that such a support will commonly be cantilevered away from the body and will accordingly, be required to be formed of stronger materials than a conventional sling which may be cumbersome and heavy. Such an orientation of the arm extending away from the body may also be prone to catching or impacting external objects thereby risking further injury to the shoulder.

### SUMMARY OF THE INVENTION

According to a first embodiment of the present invention there is disclosed an apparatus for immobilized a shoulder of a user comprising an elbow engaging member receivable on a first elbow of a user and a shoulder engaging member receivable on an opposed shoulder from the first elbow. The apparatus further comprises a first tension member extending between the elbow engaging member and the shoulder engaging member behind the back of the user, the first tension member being tightenable so as to draw the first elbow behind the frontal plane of the torso of the user and retain the elbow proximate to the torso. The apparatus further comprises a second tension member extending from a wrist corresponding to the shoulder to be immobilized, the second tension member being securable to the user so as to support and maintain the wrist proximate to the user's torso.

The second strap may extend from the wrist to the shoulder engaging member. The second strap may be securable to an article of clothing of the user. The first and second tension members may comprise straps. The straps may be formed of a material selected from the group consisting of leather, fabric, elastics, plastic or rubber.

The first and second tension members may include tightening devices for reducing a length of the first and second tension members so as to tighten the first and second tension members. The tightening devices may comprise buckles. The tightening devices may comprise selectably attachable hook and loop fasteners. The shoulder engaging member may comprise a shoulder cap receivable over the shoulder.

The elbow engaging member may comprise a tubular sleeve. The tubular sleeve may have a central passage having a curvature corresponding to the shape of the first elbow. The first tension member may be permanently secured to the shoulder engaging member and the elbow engaging member and the second tension member may be permanently secured to the shoulder engaging member. The first tension member may be selectably attachable to the shoulder engaging member and the elbow engaging member and the second tension member may be selectably attachable to the shoulder engaging member. The first tension member may be secured to the elbow engaging member proximate to the cubital fossa of the first elbow.

The apparatus may further comprise a wrist strap at a distal end of the second tension member. The apparatus may further comprise a connector at a distal end of the second tension member operable to engage an article of clothing around the wrist of the user.

According to a further embodiment of the present invention there is disclosed a method of immobilizing a shoulder of a user comprising connecting a first tension member between an elbow corresponding to the shoulder to be immobilized to an opposed shoulder of the user behind the back of the user. The method further comprises connecting a second tension member between a wrist corresponding to the shoulder to be immobilized and the user's torso so as to support and maintain the wrist proximate to the user's torso and tightening the first tension member so as to draw the elbow behind a frontal plane of the user's torso proximate to the user's torso.

According to a further embodiment of the present invention there is disclosed an apparatus for immobilizing a shoulder of a user comprising a first body strap sized to surround the torso of the user and a first arm retainer operably connected to the first body strap proximate to the side of the torso of a user, the first arm retainer being sized to surround the arm corresponding to the shoulder to be immobilized and connected to the first arm retainer. The apparatus further comprises a second body strap sized to surround the thigh of the user and a second arm retainer operably connected to the second body strap proximate to the outside of the thigh of the user, the second arm retainer being sized to surround the arm corresponding to the shoulder to be immobilized.

The first and second members may comprise body straps. The body straps may be formed a flexible material. The flexible material may be selected from the group consisting of leather, fabric, plastic or rubber.

The straps may include semi-rigid portions located to retain the first and second arm retainers proximate to a side of the user. The first and second members may include fasteners for securing first and second ends thereof together to form a continuous loop. The fasteners may comprise buckles. The fasteners may comprise hook and loop fasteners. The fasteners may comprise free distal first and second ends operable to be tied to each other.

The first and second arm fasteners comprise arm straps. The first and second arm straps may be biased around the arm of the user. The first and second arm straps may elastic. The first and second arm straps may form continuous loops. The first arm retainer may be securable proximate to the elbow of the user. The second arm member may be securable proximate to the wrist of the user.

The first and second arm retainers may have a connector therebetween. The connector may comprise a tubular member. The connector may have a frusto-conical shape. The connector may have a shape corresponding to the forearm of a user.

According to a further embodiment of the present invention there is disclosed a method of immobilizing the shoulder of a user. The method comprises securing a first member to the torso of the user and securing the elbow corresponding to the immobilized shoulder of the user to the first member in close proximity to the side of the user corresponding to the immobilized shoulder. The method further comprises securing a second member about the thigh of the user corresponding to the shoulder to be immobilized and securing the wrist corresponding to the immobilized shoulder to the second member at a location proximate to the outside of the thigh.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention wherein similar characters of reference denote corresponding parts in each view,

FIG. 1 is a front view of a user with a prior art arm sling applied to the arm of an injured shoulder.

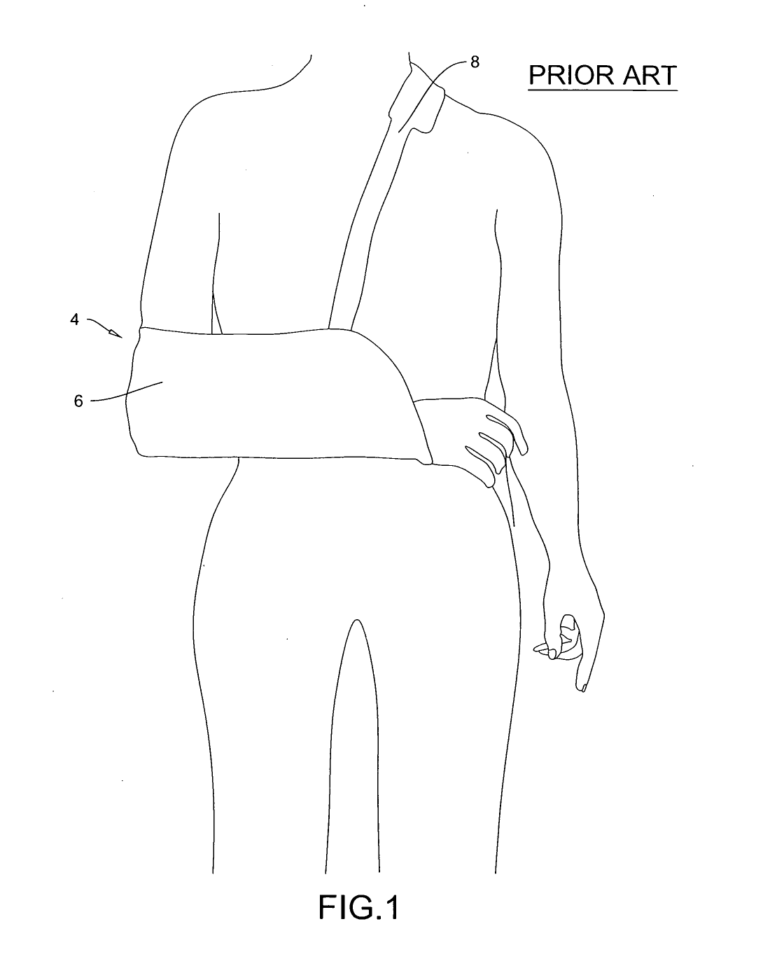

FIG. 2 is a front view of a user having an apparatus for immobilizing the shoulder according to a first embodiment of the present invention applied thereto.

FIG. 3 is a perspective view of the apparatus of FIG. 2 according to a first embodiment.

FIG. 4 is a perspective view of an apparatus for immobilizing the shoulder applied thereto according to a further embodiment.

FIG. 5 is a perspective view of the apparatus of FIG. 2 having position retaining portions.

FIG. 6 is a front perspective view of a user having an apparatus for immobilizing the shoulder according to a further embodiment of the present invention applied thereto.

FIG. 7 is a rear view of the apparatus of FIG. 6 applied to a user.

FIG. 8 is a side view of the apparatus of FIG. 6 applied to a user.

FIG. 9 is a perspective view of the apparatus of FIG. 6.

### DETAILED DESCRIPTION

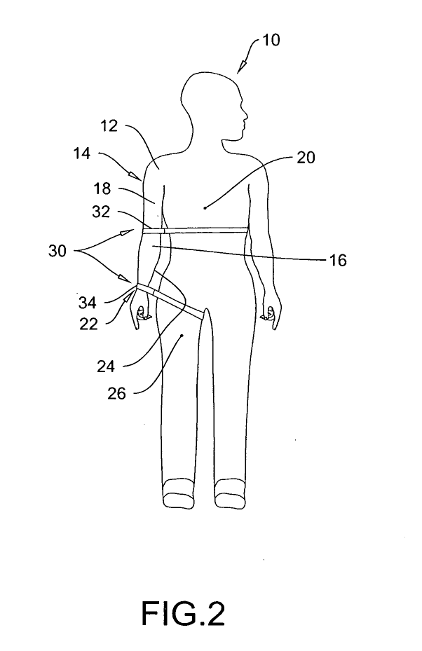

Referring to FIG. 2, an apparatus for stabilizing or immobilizing the shoulder 12 of a user 10 according to a first embodiment of the invention is shown generally at 30. The apparatus 30 is applied to the arm 14 corresponding to a shoulder 12 of a user 10 that is to be immobilized. The apparatus 30 comprises a top arm immobilizer 32 and a bottom arm immobilizer 34. As illustrated in FIG. 2, the top arm immobilizer 32 is located proximate to an elbow 16 of the shoulder 12 to be immobilized and is adapted to retain the upper arm 18 of the wearer proximate to the torso 20 of the user. The bottom arm immobilizer 34 is located proximate to the wrist 22 of the user and retains the forearm 24 of the wearer proximate to the thigh 26 of the user.

As illustrated in FIG. 2, the apparatus retains the elbow 16 proximate to the lateral side of the torso 20 corresponding to the shoulder 12 that is to be immobilized. Correspondingly the wrist 22 is also retained proximate to the outside lateral surface of the thigh 26. It will be seen in FIG. 2 that in this position, the arm 14 is retained in a substantially straight orientation generally downwardly from the shoulder 12 along the outer lateral surface of the user's body. Retaining the arm 14 of the user 10 proximate to their torso 20 maintains the shoulder 12 at a position that is more neutral and prevents internal rotation of the arm which will impede healing. Maintaining the arm 14 proximate to the torso 20 of the user 10 as shown in FIG. 2 also maintains the arm in a position that is less prone to catching or impacting external objects which could further injure the user. In particular, maintaining the wrist 22 proximate to the thigh 26 prevents the elbow from bending by a significant degree and thereafter internally rotated. The apparatus 30 is utilized to immobilizing the shoulder 12 of a user 10 such as by way of non-limiting example after they have suffered a dislocated shoulder. It will be appreciated that the apparatus 30 may be utilized for immobilizing the shoulder of the user 10 for other purposes as well such as post-operatively in shoulder surgeries. It will also be seen that securing the wrist to the thigh of the user will maintain proper alignment of the shoulder during most movements of the user such as sitting, walking and the like.

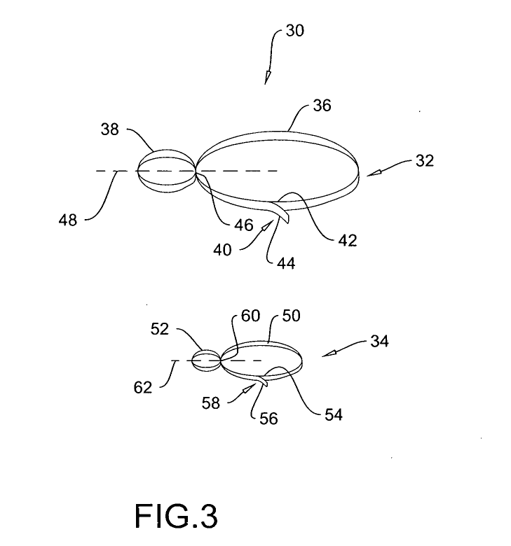

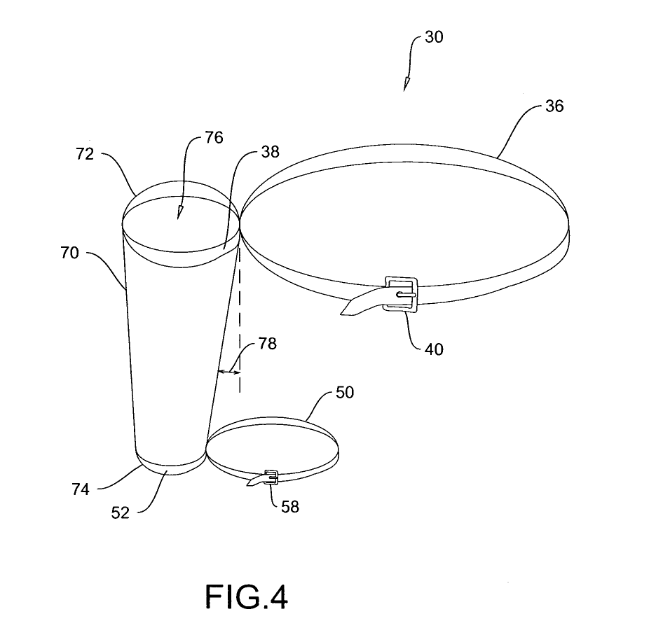

Turning now to FIGS. 3 and 4, the top arm immobilizer 32 comprises a first member 36 and an adjoining first arm retainer 38. The first member 36 may comprise a strap that is either a continuous loop or having first and second ends 42 and 44, respectively. The first and second ends 42 and 44 may be connectable to each other with a fastener 40 or the like. In particular, the first and second ends may be connected together by hook and loop fastener, a buckle as illustrated in FIG. 4 or tied to each other. It will also be appreciated that other fastener types may be utilized for connecting the first and second ends 42 and 44 of the first member 36 to each other. The first arm retainer 38 may comprise an arm strap being either formed as a continuous loop or having ends which may be connected to each other similar to the first member.

The first member 36 is sized to closely surround the torso 20 of the user. The first member 36 and first arm retainer 38 may be formed of any suitable elastic or flexible material such as, by way of non-limiting example, leather, fabrics plastics or rubbers. The first arm retainer 38 may secured to the first member 36 at a connection point 46 by any known method such as, utilizing adhesives, sewing, rivets or heat welding. In some embodiments, the first arm retainer 38 may be rotatably connected to the first member 36 about an axis generally indicated at 48 so as to permit some bending of the elbow while maintaining the elbow proximate to the lateral side of the user. Such rotation may be useful where some degree of movement of the arm is desirable such as when sitting and the like.

The bottom arm immobilizer 34 comprises a second member 50 and an adjoining second arm retainer 52. The second member 50 may comprise a strap that is either a continuous loop or having first and second ends 54 and 56, respectively. The first and second ends 54 and 56 may be connectable to each other with a fastener 58 or the like. In particular, the first and second ends may be connected together by hook and loop fastener, a buckle as illustrated in FIG. 4 or tied to each other. It will also be appreciated that other fastener types may be utilized for connecting the first and second ends 54 and 56 of the second member 50 to each other. The second arm retainer 52 may comprise an arm strap being either formed as a continuous loop or having ends which may be connected to each other similar to the first member.

The second member 50 is sized to closely surround the thigh 26 of the user. The second member 50 and second arm retainer 52 may be formed of any suitable elastic or flexible material such as, by way of non-limiting example, webbing, leather, fabrics, neoprene, plastics or rubbers. The second arm retainer 52 may secured to the second member 50 by any known method such as, utilizing adhesives, sewing, rivets or heat welding. In some embodiments, the second arm retainer 52 may be rotatably connected to the second member 50 about an axis generally indicated at 62 so as to permit some bending of the elbow while maintaining the wrist proximate to the lateral side of the user's thigh. Such rotation may be useful where some degree of movement of the arm is desirable such as when sitting and the like.

As illustrated in FIG. 4, an alternative embodiment of the invention is illustrated having a connector 70 between the first and second members 36 and 50. The connector may include the first and second arm retainers 38 and 52 and may be secured therein or may optionally be connected directly to the first and second members 36 and 50 by any known means. The connector 70 comprises a rigid or semi-rigid elongate tubular member extending between top and bottom ends 72 and 74, respectively and includes an open bore 76 therethrough. The bore 76 is sized to retain the forearm 24 of the user 10 therein so as to prevent rotation of the arm away from the torso. The may have a frusto-conical shape with the bottom end 74 being smaller than the top end 74 with a taper angle generally indicated at 78 therebetween. Optionally the bore 76 may have a shape corresponding to the shape of the forearm 24 of the user 10 so as to securely hold the forearm therein.

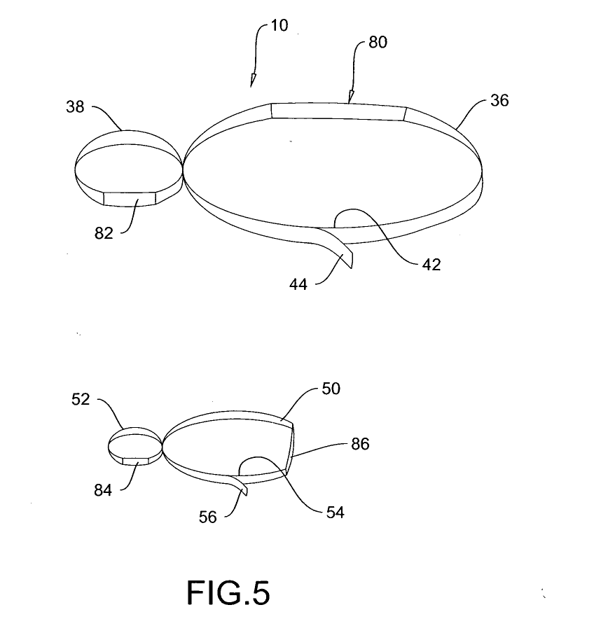

Turning no to FIG. 5, an alternative embodiment is illustrated in which the first member 36 includes a position retaining portion 80. The first member position retaining portion 80 may comprise as rigid or semi-rigid member inserted into or adhered to the first member at a position corresponding to the back of the user. Accordingly when the first member is secured around the torso of the user the first member retaining portion 80 will maintain the first member in a position such that the first arm retainer 38 is located to a lateral side of the torso of the user. Similarly, the first and second arm retainers 38 and 52 may also include first and second arm band locators, 82 and 84 corresponding to the front of the elbow of the user and the front or back of the wrist of the user so as to maintain the elbow located therein at an orientation as desired by the user, such as, by way of non-limiting example to maintain the palm of the hand facing forward. The retaining portions may be formed of any suitable material such as, leather, plastic or the like and may be selected to have a stiffer bending stiffness or thicker cross-section than the remainder of the apparatus which may remain flexible and comfortable to wear. Optionally, the second member 50 may also include a retaining portion 86 located to at a position to be maintained against a side of the leg of the user so as to maintain second arm retainer 52 at the side of the leg.

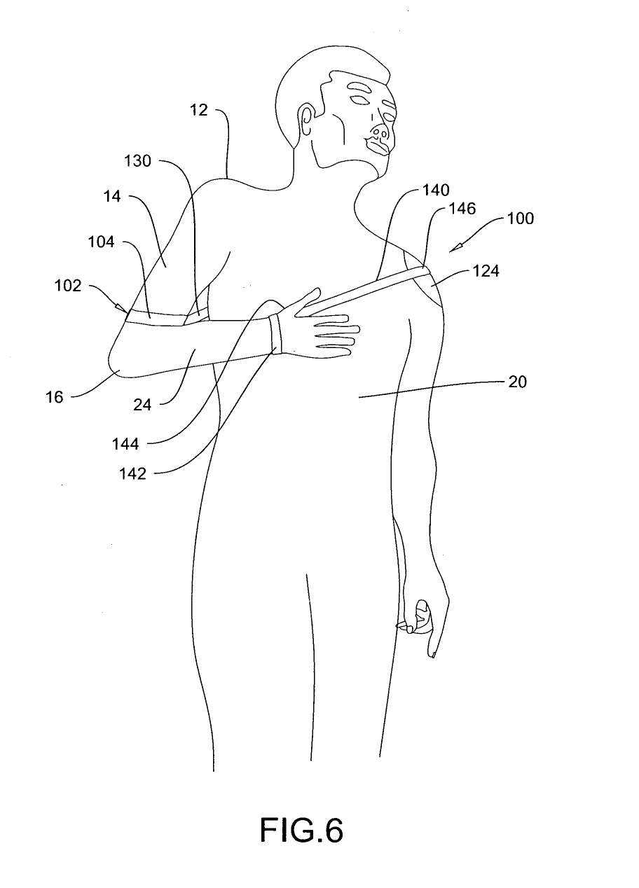

Turning now to FIGS. 6 through 9, an apparatus 100 according to an alternative embodiment of the present invention is illustrated having an elbow engaging member 102, a shoulder engaging member 120 and first and second tension members, 130 and 140 respectively. The elbow engaging member 102 is receivable on a first elbow of a user corresponding to the shoulder to be immobilized. The shoulder engaging member 120 is receivable on an opposed shoulder from the first elbow and the first tension member 130 extends between the elbow engaging member 102 and the shoulder engaging member 120 behind the back of the user. The second tension member supports and maintains the wrist proximate to the user's torso while the first tension member 130 is tightened so as to draw the elbow of a wearer behind the frontal plane of a user and thereby to maintain the shoulder to be isolated in external rotation.

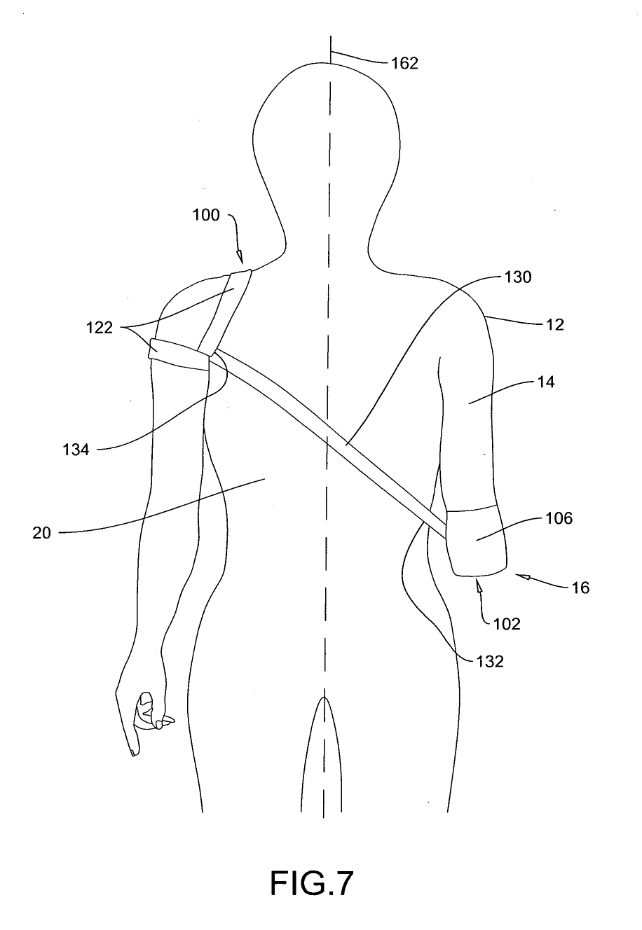

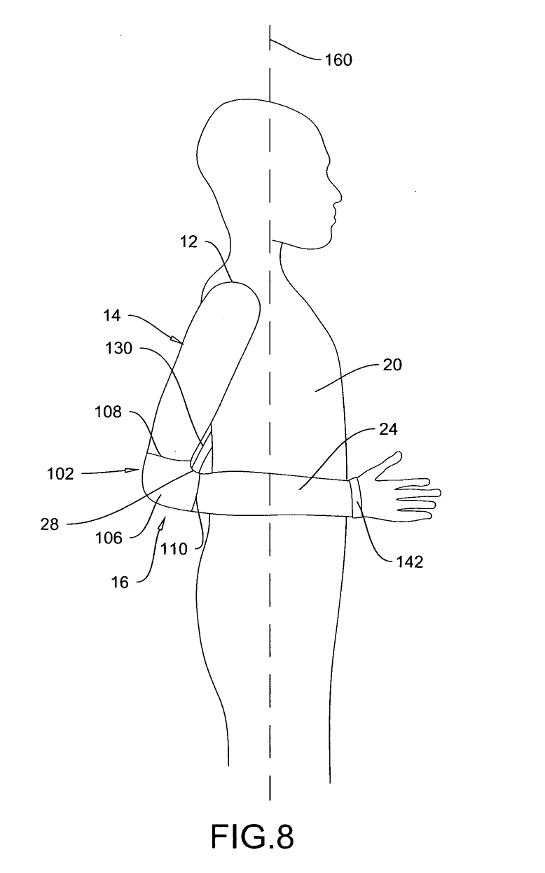

The elbow engaging member 102 may comprise an elbow strap 104 as illustrated in FIG. 6 or may optionally comprise a curved elbow sleeve 106 as illustrated in FIGS. 7 and 8. The elbow strap 104 may be sized to receive the elbow of a user therein and may be formed of a continuous loop or a selectably closable loop which is closable with hook and loop fasteners, buckles or the like. The elbow sleeve 106 may be formed to have an elbow shape with a central cavity therethrough having inlet and outlet ends, 108 and 110, respectively adapted to surround the arm of a user above and below the elbow, respectively. The elbow strap 104 or elbow sleeve 106 may be formed of any suitable material, such as, by way of non-limiting example, webbing or netting, leather, fabrics, neoprene, plastics or rubbers. It will also be appreciated that the elbow engaging member 102 may be formed of a pair of elbow straps corresponding to the inlet and outlet ends 108 and 110 of the elbow sleeve 106 such that one strap surrounds the upper arm and the other strap surround the lower arm proximate to the elbow of the user.

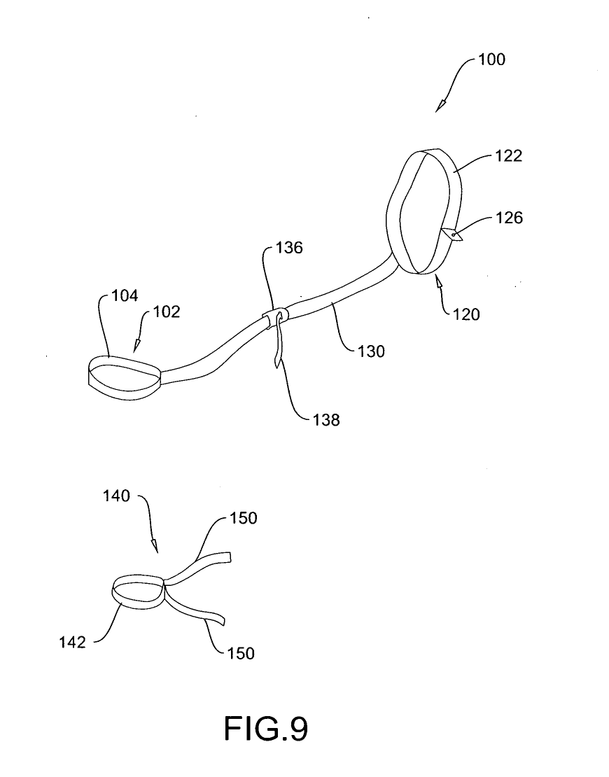

The shoulder engaging member 120 may be formed of a shoulder loop 122 as illustrated in FIG. 6 or a shoulder cap 124 as illustrated in FIG. 7. Additionally the shoulder loop 122 may be formed of a single loop as illustrated in FIG. 9 or a pair of angularly oriented loops as illustrated in FIG. 7 for extending around the top and side of the shoulder, respectively. The shoulder engaging member 120 may be formed of any suitable material, such as, by way of non-limiting example, webbing or netting, leather, fabrics, neoprene, plastics or rubbers. The shoulder loop 122 may optionally include a pull tab 126 extending therefrom for a user to grasp to facilitate pulling the loop onto their shoulder.

The first tension member 130 extends between the elbow engaging member 102 and the shoulder engaging member 120. The first tension member 130 may comprise an elongate strap having first and second ends, 132 and 134, respectively. The first end 132 is secured or connectable to the elbow engaging member 102 and the second end 134 is secured or connectable to the shoulder engaging member 120. The first tension member 130 may be permanently secured to either or both of the elbow engaging member 102 and shoulder engaging member 120 by any known means, such as, by way of non-limiting example, stitching, adhesives, fabric welding or being formed integrally therewith or may also be selectably attachable by any known means, such as, by way of non-limiting example, fasteners, buttons, clips, snaps, hook and loop fasteners or ties.

According to one embodiment, the first end 132 may be attachable or affixed to the elbow engaging member 102 at the cubital fossa 28 of the elbow 16 as illustrated in FIG. 8. Similarly, the second end 134 may be secured to a rearmost portion of the shoulder engaging member 120 or proximate to the rear of the armpit of the opposed shoulder as illustrated in FIG. 7. The first tension member 130 has a length selected to retain the elbow behind the frontal plane 160 of a user and may optionally also be retained behind the back of the user as illustrated in FIG. 8. Similarly, the first tension member 130 is also selected to retain the elbow 16 proximate to the torso 20 of the user such that the forearm 24 is substantially parallel to the median plane 162 of the user. It will be appreciated that maintaining tension on the elbow 16 in a direction towards the opposed shoulder behind the back of the user will prevent the elbow from moving away from the body and thereby prevent the shoulder from internally rotating.

The first tension member 130 may be formed of any suitable material, such as, by way of non-limiting example, webbing or netting, leather, fabrics, neoprene, plastics or rubbers. Optionally the first tension member 130 may be formed of an elastic material, such as an elasticized fabric such that the first tension member 130 will provide some degree of movement for the user while maintaining a biasing force towards the opposite shoulder behind the back as described above. In other embodiments, the first tension member 130 may have a tightening device, such as, by way of non-limiting example, a buckle 136, such as a slide release buckle, belt-style buckle, friction buckle or the like having a free end 138 which may be grasped by a user so as to shorten the first tensioning member to apply the desired amount of tension thereto. The first tension member may optionally be formed as two strap sections having mating hook and loop fasteners therebetween so as to permit connection at one of a plurality of locations.

The second tension member 140 extends from the wrist 22 of the user so as to maintain the wrist proximate to the torso 20 of the user. The second tension member 140 may include a wrist loop 142 at a first end thereof sized to receive the wrist 22 of the user therein. As illustrated in FIG. 6, the second tension member 140 may comprise an elongate strap having first and second ends, 144 and 146, respectively. The first end 144 is secured or connectable to the wrist loop 142 and the second end 146 is secured or connectable to the shoulder engaging member 120. The second tension member 140 may be permanently secured to either or both of the wrist loop 142 and shoulder engaging member 120 by any known means, such as, by way of non-limiting example, stitching, adhesives, fabric welding or being formed integrally therewith or may also be selectably attachable by any known means, such as, by way of non-limiting example, fasteners, buttons, clips, snaps, hook and loop fasteners or ties.

Optionally, the second tension member 140 may secure the wrist 22 to a portion of the user's clothing, such as, by way of non-limiting example a belt or belt loop as illustrated in FIG. 9. In such embodiments, the second tension member 140 may comprise a pair of opposed straps 150 extending from the wrist loop 142 which are operable to be passed through the belt or belt loop of a user and thereafter secured to each other through the use of hook and loop fasteners, ties or the like.

According to one embodiment, the second end 146 may be attachable or affixed to the frontmost portion of the shoulder engaging member 120 or proximate to the front of the armpit of the opposed shoulder as illustrated in FIG. 6. The second tension member 140 has a length selected to retain the wrist in front of the frontal plane 160 of a user and may optionally also be retained in front of the torso of the user as illustrated in FIG. 8. Similarly, the second tension member 140 is also selected to retain the wrist 22 proximate to the torso 20 of the user such that the forearm 24 is substantially parallel to the median plane 162 of the user and to support the wrist of the user so as to maintain the elbow 16 in a bent position. It will be appreciated that maintaining the elbow 16 in a bent position will prevent rotation of the writs 22 from being translated into shoulder rotation as would be possible with a straight arm. It will also be appreciated that maintaining the wrist proximate to the torso 20 will prevent the shoulder from externally rotating further than the orientation at which the forearm 24 is substantially parallel with the median pane 162 of the user.

The second tension member 140 may be formed of any suitable material, such as, by way of non-limiting example, webbing or netting, leather, fabrics, neoprene, plastics or rubbers. Optionally the second tension member 140 may be formed of an elastic material, such as an elasticized fabric such that the second tension member 140 will provide some degree of movement for the user while maintaining a biasing force to retain the wrist 22 proximate to the torso 20 of the user. In other embodiments, the second tension member 140 may have a tightening device similar to the tightening device of the first tension member 130 as described above.

While specific embodiments of the invention have been described and illustrated, such embodiments should be considered illustrative of the invention only and not as limiting the invention as construed in accordance with the accompanying claims.

What is claimed is:

1. An apparatus for immobilized a shoulder of a user comprising:
an elbow engaging member receivable on a first elbow of a user;
a shoulder engaging member receivable on an opposed shoulder from said first elbow;
a first tension member extending between said elbow engaging member and said shoulder engaging member behind the back of the user, said first tension member being tightenable so as to draw said first elbow behind the frontal plane of the torso of the user and retain said elbow proximate to the torso; and
a second tension member extending from a wrist corresponding to the shoulder to be immobilized, said second tension member being securable to the user so as to support and maintain said wrist proximate to the user's torso.

2. The apparatus of claim 1 wherein said second strap extends from said wrist to said shoulder engaging member.

3. The apparatus of claim 1 wherein said second strap is securable to an article of clothing of the user.

4. The apparatus of claim 1 wherein said first and second tension members comprise straps.

5. The apparatus of claim 4 wherein said straps are formed of a material selected from the group consisting of leather, fabric, elastics, plastic or rubber.

6. The apparatus of claim 1 wherein said first and second tension members include tightening devices for reducing a length of said first and second tension members so as to tighten said first and second tension members.

7. The apparatus of claim 6 wherein said tightening devices comprise buckles.

8. The apparatus of claim 6 wherein said tightening devices comprise selectably attachable hook and loop fasteners.

9. The apparatus of claim 1 wherein said shoulder engaging member comprises a shoulder cap receivable over the shoulder.

10. The apparatus of claim 1 wherein said elbow engaging member comprises a tubular sleeve.

11. The apparatus of claim 10 wherein said tubular sleeve has a central passage having a curvature corresponding to the shape of said first elbow.

12. The apparatus of claim 1 wherein said first tension member is permanently secured to said shoulder engaging member and said elbow engaging member and said second tension member is permanently secured to said shoulder engaging member.

13. The apparatus of claim 1 wherein said first tension member is selectably attachable to said shoulder engaging member and said elbow engaging member and said second tension member is selectably attachable to said shoulder engaging member.

14. The apparatus of claim 1 wherein said first tension member is secured to the elbow engaging member proximate to the cubital fossa of said first elbow.

15. The apparatus of claim 1 further comprising a wrist strap at a distal end of said second tension member.

16. The apparatus of claim 1 further comprising a connector at a distal end of said second tension member operable to engage an article of clothing around said wrist of the user.

17. A method of immobilizing a shoulder of a user, the method comprising:
connecting a first tension member between an elbow corresponding to the shoulder to be immobilized to an opposed shoulder of the user behind the back of the user;
connecting a second tension member between a wrist corresponding to the shoulder to be immobilized and the user's torso so as to support and maintain said wrist proximate to the user's torso; and
tightening said first tension member so as to draw said elbow behind a frontal plane of the user's torso proximate to the user's torso.

18. An apparatus for immobilized a shoulder of a user comprising:
a first body strap sized to surround the torso of the user;
a first arm retainer operably connected to said first body strap proximate to the side of the torso of a user, said first arm retainer being sized to surround the arm corresponding to the shoulder to be immobilized and connected to said first arm retainer;
a second body strap sized to surround the thigh of the user; and
a second arm retainer operably connected to said second body strap proximate to the outside of the thigh of the user, said second arm retainer being sized to surround the arm corresponding to the shoulder to be immobilized.

19. The apparatus of claim 16 wherein said first and second arm retainers have a connector therebetween comprising a tubular member having a shape corresponding to the forearm of a user.

20. A method of immobilizing the shoulder of a user, the method comprising:
securing a first member to the torso of the user;
securing the elbow corresponding to the immobilized shoulder of the user to said first member in close proximity to the side of the user corresponding to the immobilized shoulder;
securing a second member about the thigh of the user corresponding to the shoulder to be immobilized; and
securing the wrist corresponding to the immobilized shoulder to said second member at a location proximate to the outside of said thigh.
